(19) United States
(12) Patent Application Publication
(10) Pub. No.: US 2026/0085337 A1
(43) Pub. Date: Mar. 26, 2026

(54) ENGINEERED BACTERIA TO PIGMENT POLYHYDROXYALKANOATES

(71) Applicant: NEOX Public Benefit LLC, New York, NY (US)

(72) Inventors: Neri Oxman, New York, NY (US); Kenneth Walker, New York, NY (US); Andrea H. Westlie, New York, NY (US); Finn Stirling, Williston Park, NY (US)

(21) Appl. No.: 19/340,192

(22) Filed: Sep. 25, 2025

Related U.S. Application Data
(60) Provisional application No. 63/698,824, filed on Sep. 25, 2024.

(51) Int. Cl. C12P 17/16; B29B 11/10; B29C 64/118; B29K 67/00; B33Y 30/00; B33Y 40/00; B33Y 70/00

(52) U.S. Cl. CPC C12P 17/165; B33Y 70/00; B29B 11/10; B29C 64/118; B29K 2067/04; B33Y 30/00; B33Y 40/00

(57) ABSTRACT

In general, the present disclosure is generally directed to a method for producing a filament. The method includes providing an engineered bacterial culture comprising a pigment. Additionally, the method includes extracting the pigment from the engineered bacterial culture by contacting the engineered bacterial culture with a solvent mixture.

Also, the method includes mixing the extracted pigment with a polyhydroxyalkanoate (PHA) to form a pigment PHA mixture. Further, the method includes extruding the pigment PHA mixture as a filament.

### CROSS REFERENCE TO RELATED APPLICATIONS

This application claims the filing benefit of U.S. Provisional Patent Application Ser. No. 63/698,824, filed on Sep. 25, 2024, which is incorporated herein by reference.

### FIELD

The present disclosure relates generally to engineering bacteria to pigment thermoplastic polymers. More particularly, the present disclosure relates to methods for producing pigmented thermoplastic polymers for three-dimensional (3D) printing.

### BACKGROUND

Many industrial pigments and dyes are derived from petrochemical feedstocks, a practice that has significant environmental consequences. The extraction and processing of these materials contribute to air and water pollution, including the release of greenhouse gases and toxic byproducts that can harm ecosystems. Petrochemical-based production also relies on non-renewable fossil fuels, further depleting natural resources and exacerbating environmental degradation. This reliance on fossil-based materials presents a major sustainability challenge for industries that depend on pigments and dyes.

In addition to environmental concerns, the production of petrochemical-based pigments is deeply embedded in complex global supply chains. These supply chains are vulnerable to disruptions, such as geopolitical conflicts, transportation delays, and price fluctuations in oil markets. This dependence on global logistics not only increases the carbon footprint associated with long-distance transport but also introduces instability in the availability and cost of these materials. As a result, there is a growing need to explore alternative, more sustainable sources of pigments and dyes that reduce environmental impact.

### SUMMARY

In general, the present disclosure is directed to a method for producing a filament. The method includes providing an engineered bacterial culture comprising a pigment. Additionally, the method includes extracting the pigment from the engineered bacterial culture by contacting the engineered bacterial culture with a solvent mixture. Also, the method includes mixing the extracted pigment with a polyhydroxyalkanoate (PHA) to form a pigment PHA mixture. Further, the method includes extruding the pigment PHA mixture as a filament.

In some instances, the bacterial culture may be a strain selected from a group consisting of Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinomycetemcomitans, cyanobacteria, Escherichia coli, Helicobacter pylori, Selenomonas ruminantium, Shigella sonnei, Zymomonas mobilis, Mycoplasma mycoides, Treponema denticola, Bacillus thuringiensis, Staphylococcus lugdunensis, Leuconostoc oenos, Coryne bacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus cereus, Bacillus popilliae, Synechocystis strain PCC6803, Bacillus liquefaciens, Pyrococcus abyssi, Selenomonas ruminantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis.

In some instances, the bacterial culture may be an E. coli strain.

In some instances, the pigment may be indigo.

In some instances, the pigment may be indirubin.

In some instances, the pigment may be melanin.

In some instances, the solvent may be water, acetone, petroleum, ether, chloroform, ethanol, ethyl acetate, or a combination thereof.

In some instances, the solvent may be chloroform.

In some instances, the solvent mixture may include the pigment and a solvent at a ratio of from about 1:5 to about 5:1.

In some instances, the polymer may be a polyhydroxyalkanoate (PHA).

In some instances, the PHA may be selected from a group consisting of 3-hydroxypropionate (3HP), 3-hydroxybutyrate (3HB), 3-hydroxyvalerate (3HV), 3-hydroxyhexanoate (3HHx), 3-hydroxyoctanoate (3HO), 3-hydroxydecanoate (3HD), 3-hydroxy-5-phenylvalerate (3HPV), 4-hydroxybutyrate (4HB) and 4-hydroxyvalerate, 4-hydroxybutyrate (4HB), 4-hydroxyvalerate (4HV), or a combination thereof.

In some instances, the PHA may be selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

In some instances, the PHA may be a mixture of a first polymer and a second polymer in a ratio of from about 1:5 to about 5:1.

In some instances, the PHA may be a mixture of poly(3-hydroxybutyrate-co-4-hydroxyvalerate) (P(3HB-co-4HB)) and poly(3-hydroxybutyrate-co-3-hydroxyvalerate) (P(3HB-co-3HV)).

Example aspects of the present disclosure provide a three dimensional (3D) printing system. The system includes a supply source including, but not limited to, a pigment polyhydroxyalkanoate (PHA) mixture. The pigment PHA mixture may include, but is not limited to, an extracted pigment produced from an engineered bacteria cell mixed with a PHA. Additionally, the system may include a printer head that is configured to receive the polymer composition from the supply source and deposit the pigment PHA mixture onto a substrate.

In some instances, the supply source may be a printer cartridge. The printer cartridge may be a filament, which may include the pigment PHA mixture.

In some instances, the supply source may be a hopper. The hopper may be a pellet, which may include the pigment PHA mixture.

In some instances, the substrate may be an object.

In some instances, the object may be selected from a group consisting of footwear, apparel, sports equipment, wearable devices, upholstery, jewelry, and cosmetics.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 depicts representative plasmid maps for E. coli Gibson_puc19_indigo and Gibson_puc19_tyr1.

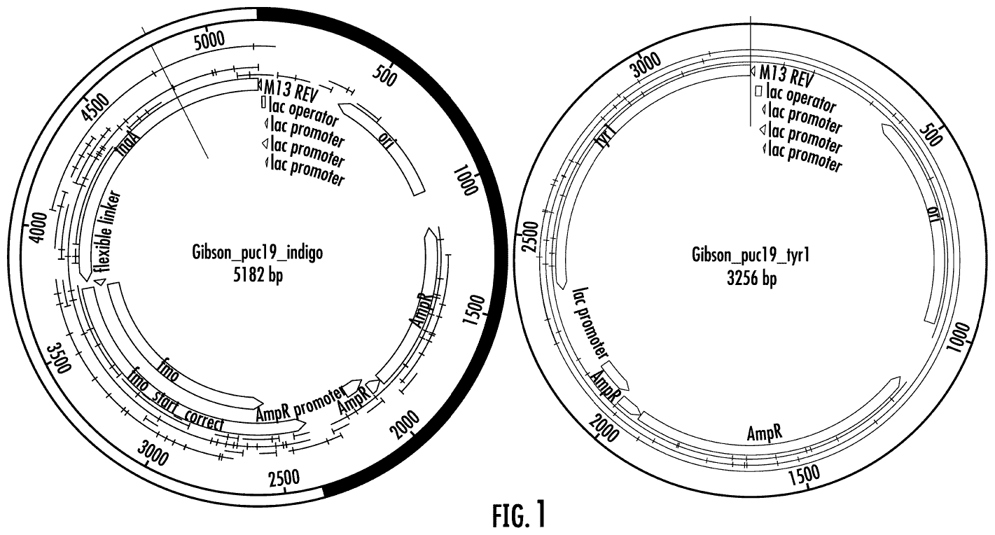

FIG. 2 depicts a flow diagram of an exemplary method for producing a filament.

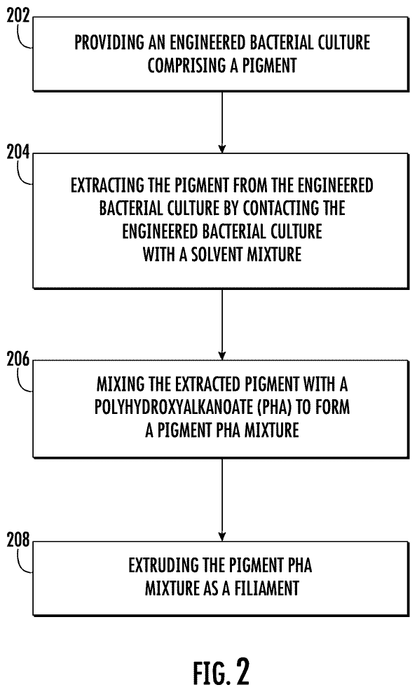

FIG. 3 depicts a 3D printer system.

FIG. 4 depicts a flow diagram of an exemplary method for producing and purifying pigment PHA.

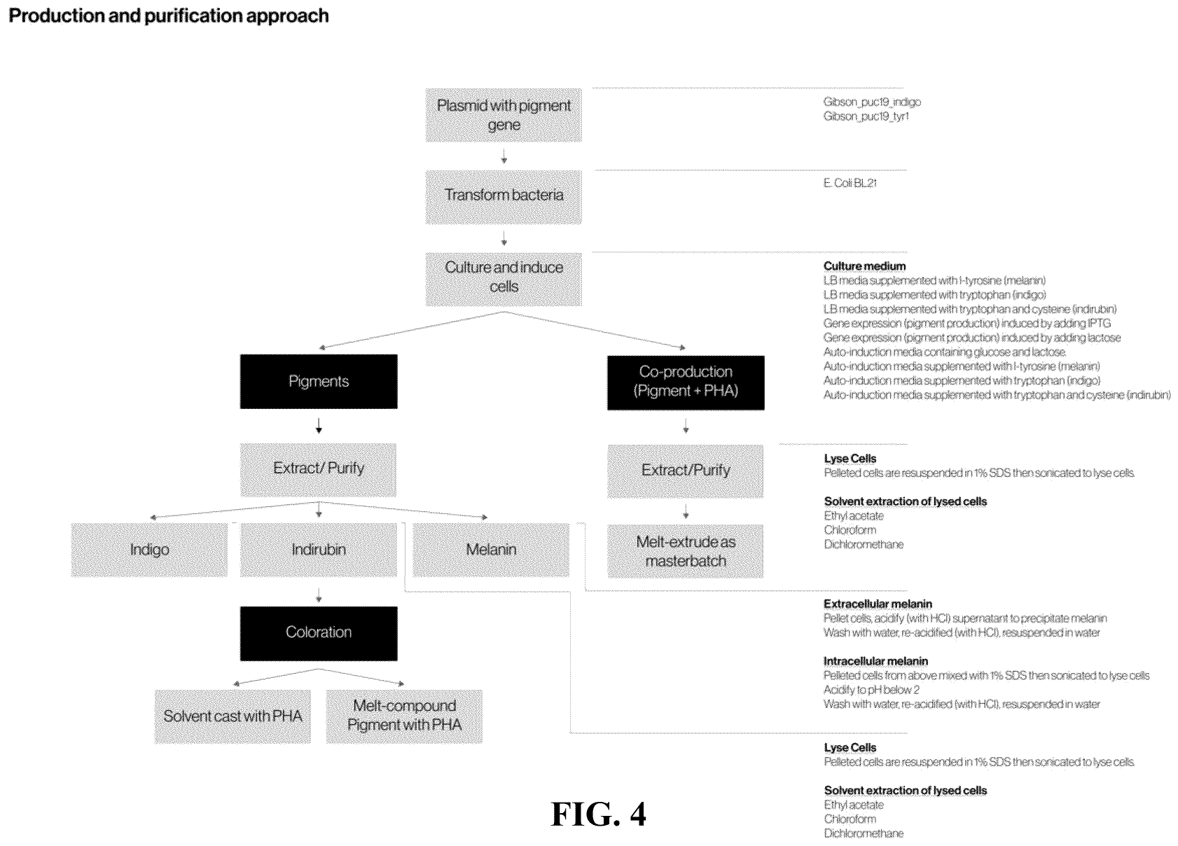

FIG. 5 depicts thin layer chromatography of indigo and indirubin cultures.

FIG. 6 depicts dependency of melanin production from E. coli ₚGibson_puc_tyr1 on tyrosine concentration.

FIG. 7 depicts a flow diagram of an exemplary method of direct bacterial application of pigment PHA to a textile.

FIG. 8 depicts a timelapse of direct bacterial pigmentation. Socks were soaked in induced cultures of either E. coli pGibson_puc19_indigo or E. coli pGibson_puc19_tyr1 and imaged over time as pigmentation develops.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to compositions and methods for producing pigments using engineered bacteria cells for three-dimensional (3D) printing.

Beneficially, pigments disclosed herein are produced via environmentally friendly conditions from simple and abundant feedstocks such as sugars and waste food streams in temperate conditions through enzymatic processes. Methods disclosed herein may provide 3D printing methods using natural pigments mixed with polyhydroxyalkanoate to form products or objects that are ecologically friendly.

In some example embodiments, pigments disclosed herein may be produced using genetically engineered bacterium. For instance, the genetically engineered bacterium may include a genetically engineered organism. The genetically engineered organism may be a bacterial cell. In another example embodiment, the genetically engineered organism may be a mammalian or yeast cell.

In one example embodiment, the bacterial cell may be selected from a group consisting of Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinomycetemcomitans, cyanobacteria, Escherichia coli, Helicobacter pylori, Selenomonas ruminantium, Shigella sonnei, Zymomonas mobilis, Mycoplasma mycoides, Treponema denticola, Bacillus thuringiensis, Staphylococcus lugdunensis, Leuconostoc oenos, Coryne bacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus cereus, Bacillus popilliae, Synechocystis strain PCC6803, Bacillus liquefaciens, Pyrococcus abyssi, Selenomonas ruminantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. For instance, in some example embodiments, the bacterial cell may be Escherichia coli.

In another example embodiment, the bacterial cell may be selected from a group consisting of Yersinia spp., Escherichia spp., Klebsiella spp., Acinetobacter spp., Bordetella spp., Neisseria spp., Aeromonas spp., Francisella spp., Coryne bacterium spp., Citrobacter spp., Chlamydia spp., Haemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Streptomyces spp., Bacteroides spp., Prevotella spp., Clostridium spp., Bifidobacterium spp., or Lactobacillus spp.

In some example embodiments, indigo may be produced from the engineered bacterial cell disclosed herein. For instance, indigo may be produced from engineered E. coli disclosed herein. The production of indigo in an engineered E. coli cell involves the modification of key biosynthetic pathways to produce the blue dye through natural enzymatic processes. The engineered E. coli cell may be modified to express specific enzymes that convert intracellular tryptophan into indole, the primary precursor for indigo synthesis. This is achieved by overexpressing the enzyme tryptophanase (TnaA), which catalyzes the conversion of tryptophan into indole. Once indole is formed, it is further oxidized into indoxyl by oxygenases, such as flavin-containing monooxygenases (FMOs) or cytochrome P450 enzymes. Upon exposure to atmospheric oxygen, indoxyl spontaneously dimerizes to form the blue dye indigo.

In some example embodiments, indirubin may be produced from the engineered bacterial cell disclosed herein. For instance, indirubin may be produced from an engineered E. coli cell disclosed herein. The production of indirubin in an engineered E. coli cell involves the modification of key metabolic pathways to biosynthesize indirubin, a bioactive isomer of indigo. The process begins by expressing enzymes that convert intracellular tryptophan into indole via the enzyme tryptophanase (TnaA) in the engineered E. coli cell. Indole is then oxidized to indoxyl by introducing oxygenase enzymes such as cytochrome P450s or flavin-containing monooxygenases (FMOs). In a controlled environment, indoxyl undergoes oxidative dimerization, leading to the formation of indirubin as the final product.

In some example embodiments, melanin may be produced from the engineered bacterial cell disclosed herein. For instance, melanin may be produced from engineered E. coli disclosed herein. Melanin may be synthesized from tyrosine through the activity of the enzyme tyrosinase. In the engineered E. coli cell, genes encoding tyrosinase, along with other supporting enzymes, may be introduced and expressed to catalyze the conversion of intracellular tyrosine into dopaquinone, a key intermediate. This intermediate undergoes a series of non-enzymatic polymerization reactions, resulting in the production of melanin, which accumulates within or outside the cell.

In some example embodiments, bacterial cells may be transformed with a DNA construct that includes genes for producing a pigment of interest. The DNA construct may be incorporated into a vector for transformation into bacterial cells. As used herein, “transformation”refers to the process of introducing heterologous DNA into a host cell. Transformation may be carried out utilizing transformation techniques well understood in the art.

For instance, the expression vectors may include, but are not limited to, a transcriptional initiation region linked to the nucleic acid sequence that encodes the enzyme; a plurality of restriction sites for insertion of the DNA to be under the transcriptional regulation of various control elements; and selectable marker genes. Suitable control elements such as enhancers/promoters, splice junctions, polyadenylation signals, etc. may be placed in close proximity to the coding region to allow for initiation of transcription and/or correct processing of the primary transcript, i.e., the coding region for the enzyme. Alternatively, the coding region utilized in an expression vector may contain endogenous enhancers/promoters, splice junctions, intervening sequences, polyadenylation signals, etc., or a combination of both endogenous and exogenous control elements.

In general, an expression vector generally may include in the 5′-3′ direction of transcription, a promoter, a transcriptional and translational initiation region, a DNA sequence that encodes the enzyme, and a transcriptional and translational termination region functional in the host cell.

In general, any suitable promoter may be used that is capable of operative linkage to the heterologous DNA such that transcription of the DNA may be initiated from the promoter by an RNA polymerase that may specifically recognize, bind to, and transcribe the DNA in an open reading frame. For instance, a promoter may include a constitutive promoter, inducible promoter, regulated promoter, cell specific promoter, viral promoter, synthetic promoter, or a combination thereof.

A promoter may be obtained from a variety of different sources. For instance, a promoter may be derived from a native gene of the host cell, be composed of different elements derived from different promoters found in nature, or be composed of nucleic acid sequences that are entirely synthetic. A promoter may be derived from many different types of organisms and tailored for use within a given cell. For example, a promoter may include regions to which other regulatory proteins may bind in addition to regions involved in the control of the protein translation, including coding sequences.

A translation initiation sequence can be derived from any source, e.g., any expressed E. coli gene. Generally, the gene is a highly expressed gene. A translation initiation sequence can be obtained via standard recombinant methods, synthetic techniques, purification techniques, or combinations thereof, which are all well known.

The termination region may be native with the transcriptional initiation region, may be native with the coding region, or may be derived from another source. Transcription termination sequences recognized by the transformed cell are regulatory regions located 3′ to the translation stop codon, and thus together with the promoter flank the coding sequence. Examples include transcription termination sequences derived from genes with strong promoters, such as the trp gene in E. coli as well as other biosynthetic genes.

Vectors that may be used include, but are not limited to, those able to be replicated in prokaryotes and eukaryotes. For example, vectors may be used that are replicated in bacteria, yeast, insect cells, and mammalian cells. Examples of vectors include plasmids, phagemids, bacteriophages, viruses (e.g., baculovirus), cosmids, and F-factors. The vector may, if desired, be a bi-functional expression vector that may function in multiple hosts. In some example embodiments, the vector may be a plasmid.

In some example embodiments, bacteria may be used as host cells. Examples of bacteria include, but are not limited to, Gram-negative and Gram-positive organisms. For instance, an E. coli expression system suitable for T7 protein expression may be used. Examples of T7 expression strains may include, without limitation, BL21(DE3), BL21(DE3)pLysS, BLR(DE3)pLysS, Tuner(DE3)pLysS, Tuner(DE3), Lemo21(DE3), NiCO2(DE3), Oragami2(DE3), Origami B(DE3), Shuffle T7 Express, HMS174(DE3), HMS174(DE3)pLysS, DH5aplhaE, Rosetta2(DE3), Rosetta2(DE3)pLysS, NovaBlue(DE3), Rosetta-gami B, Rosetta-gami B(DE3), Rosetta-gami B(DE3)pLysS, Rosetta Blue (DE3), Novagen(DE3), or Novagen(DE3)pLysS. In one example embodiment, the T7 expression strain may be BL21.

An expression vector may be introduced into bacterial cells by commonly used transformation/infection procedures. A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrating vectors may also contain bacteriophage or transposon sequences. Extrachromosomal and integrating vectors may contain selectable markers to allow for the selection of bacterial strains that have been transformed.

For instance, transformation methods may include, but are not limited to, electroporation, use of a bacteriophage, ballistic transformation, calcium phosphate co-precipitation, spheroplast fusion, electroporation, treatment of the host cells with lithium acetate or by electroporation. Transformation procedures usually vary with the bacterial species to be transformed.

Following transformation or transfection of DNA into a bacterial cell, the bacterial cell may be selected for the presence of the nucleic acid through use of a selectable marker. A selectable marker is generally encoded on the nucleic acid being introduced into the recipient cell. However, co-transfection of selectable marker may also be used during introduction of nucleic acid into a host cell. Selectable markers that may be expressed in the recipient host cell may include, but are not limited to, genes that render the recipient host cell resistant to drugs such as actinomycin Cl, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. Selectable markers may also include biosynthetic genes, such as those in the tryptophan, tyrosine, and/or cysteine biosynthetic pathways. Upon transfection or transformation of a host cell, the cell is placed into contact with an appropriate selection agent.

The resulting transformed, engineered bacterial cell disclosed herein may be utilized in methods for producing a pigment or a polyhydroxyalkanoate. Following transformation of the engineered bacterial cell, the bacterial cells may be cultured so that the bacterial cells multiply. For instance, a culture medium may include, but are not limited to, a source of carbon, salts (e.g. salts that contain magnesium, nitrogen, phosphorus, or sulfur) to allow the bacteria to synthesize protein and nucleic acids, or to activate enzymes; amino acids; vitamins, or trace elements.

Various amino acids may be utilized in a culture medium to support cell growth, protein synthesis, and biosynthetic processes. For instance, glutamine may serve as a critical energy source and nitrogen donor, playing an essential role in nucleotide and amino acid biosynthesis. Lysine is involved in various metabolic pathways and is essential for protein synthesis. Branched-chain amino acids, such as leucine, valine, and isoleucine, are crucial for cellular growth, protein production, and metabolic regulation. Also, tyrosine plays a key role in protein synthesis and serves as a precursor to several important biomolecules. Tyrosine is essential for the production of neurotransmitters such as dopamine, epinephrine, and norepinephrine, as well as for the synthesis of thyroid hormones and melanin. In a culture medium, tyrosine supports cellular functions related to these biosynthetic pathways and is particularly important in systems where melanin production or tyrosine-derived metabolic activities are involved. Tryptophan supports growth and regulates various metabolic pathways, particularly those involved in the biosynthesis of these signaling molecules and coenzymes.

Culture mediums disclosed herein may include various amino acids. For instance, the culture medium may include, but is not limited to, tyrosine, tryptophan, lysine, valine, glutamine, leucine, isoleucine, etc. In some example embodiments, the culture medium may include tyrosine for the production of melanin.

In another example embodiment, the culture medium may include tryptophan. Tryptophan can be particularly important for producing indole-based compounds like indigo or indirubin. Its inclusion in the medium is crucial for proper cellular function, growth, and secondary metabolite production. Tryptophan is able to be metabolized and used as a carbon source. Many bacteria possess this pathway, starting with the enzyme tryptophanase. Tryptophanase cleaves tryptophan, creating indole, pyruvate, and ammonium. Pyruvate can also be a source of carbon from tryptophan and is further transformed into acetyl-CoA to fuel cellular respiration. Indole is a byproduct of this reaction, accumulating in the growth medium and acting as a signal molecule of this pathway. When a monooxygenase, e.g., an aromatic monooxygenase, is expressed by the cell, indole undergoes rapid oxidation to form indoxyl, an oxidized intermediate. Indoxyl is highly reactive with itself and undergoes spontaneous dimerization to form indigoid dyes. Indigo is one dye that can be formed by this process. Indirubin can also form as a result of non-selective oxidation of the 3^{rd} and 2^{nd} carbons of indole.

In another example embodiment, the culture medium may include, but are not limited to, a byproduct or waste stream from a food production process, a fermentation process, and combinations thereof.

In yet another example embodiment, the culture medium may include, but are not limited to, M9 salts, Na₂HPO₄, KH₂PO₄, NH₄Cl, NaCl, carbon sources, glucose, sucrose, glycerol, succinate, minerals, MnSO₄, CaCl₂, ZnSO₄, CuSO₄, CoCl₂, (NH₄)₆Mo₇O₂₄, vitamins, p-aminobenzoic acid, niacinamide, DL-pantothenic acid, calcium salt, pyridoxal HCl, pyridoxamine (HCl)₂, pyridoxine HCl, riboflavin, thiamine HCl, d-biotin, folic acid, purines and pyrimidines, adenine sulfate, uracil, guanine, xanthine, amino acids, L-Alanine, L-Arginine, L-Asparagine, L-Aspartic acid, L-Cysteine, L-Glutamic acid, Glycine, L-Histidine, L-Isoleucine, L-Leucine, L-Lysine, DL-Methionine, L-Phenylalanine, L-Proline, L-Serine, L-Threonine, L-Tryptophan, L-Tyrosine, L-Valine, MgSO4, FeSO4, casamino acids, yeast extract, or a combination thereof.

To produce a pigment, the transformed bacterial cells may be cultured for a sufficient time to produce the desired product. For instance, the transformed bacterial cell may be incubated until the pigment producing strain reaches an optical density (OD₆₀₀) of 0.5 or more, such as 0.55 or more, such as 0.6 or more. For instance, the transformed bacterial cell may be incubated until the pigment producing strain reaches an optical density (OD₆₀₀) of 1 or less, such as 0.95 or less, such as 0.9 or less, such as 0.85 or less.

Once the bacterial cell has reached an optical density, the bacterial cell may subsequently be induced and cultured for a sufficient time to allow development of the pigment and/or PHA. For instance, the bacterial cell may be cultured for about 12 hours to about 48 hours, such as from about 24 hours to about 36 hours, or any range therebetween.

In some example embodiments, the pigment may be collected, separated from the bacterial cells (e.g., lysed or intact), and/or purified through any technique known in the art such as, for example, precipitation, centrifugation, filtration, and the like. In another example embodiment, the pigment may be purified via microfiltration to remove impurities resulting in an isolated pigment produced from the engineered bacterial cell disclosed herein.

Aqueous extraction may be used for water-soluble pigments where the produced pigment is powdered, soaked in water, boiled, and filtered. In another example embodiment, acid and alkali extraction may be used with glycosides, for instance. Alternatively, microwaves and ultrasonic waves may be used to accelerate extraction of pigments in aqueous solutions with lower temperature, time, and water usage. Bio-enzymes may be used to accelerate the extraction by fermentation of the pigment such as indigo or melanin. For instance, solvents including, but not limited to, acetone, ethyl acetate, petroleum, ether, chloroform, and/or ethanol may also be used as a low-water and low-temperature (such as an extraction temperature of about 45° C.) alternative to aqueous extraction (such as an extraction temperature of about 80° C.). In one example embodiment, the solvent may be chloroform. In another example embodiment, the solvent may be ethyl acetate.

Regardless of the solvent utilized in the extraction step, the pigment and solvent may form a solvent mixture. For instance, the pigment and solvent may be present in the solvent mixture at a ratio of from about 1:5 to about 5:1, such as from about 1:3 to about 3:1, or any range therebetween. In one example embodiment, the pigment and solvent may be present in the solvent mixture at a ratio of about 1:1.

In some example embodiments, plastics to be molecularly tagged may be polyhydroxyalkanoates (PHAs). PHAs are a family of biodegradable, biocompatible, and biomanufacturable polyesters. PHAs may be produced in nature by bacterial fermentation of sugar or lipids. Also, PHAs may be synthetically produced. More than 100 different monomers may be combined within this family to produce materials. PHAs have properties similar to plastics such as polypropylene due to their high molecular mass.

Examples of monomer units that may be incorporated in polyhydroxyalkanoate polymers include, but are not limited to, 2-hydroxybutyrate, glycolic acid, 3-hydroxybutyrate, 3-hydroxypropionate, 3-hydroxyvalerate, 3-hydroxyhexanoate, 3-hydroxyheptanoate, 3-hydroxyoctanoate, 3-hydroxynonanoate, 3-hydroxydecanoate, 3-hydroxydodecanoate, 4-hydroxybutyrate, 4-hydroxyvalerate, 5-hydroxyvalerate, and 6-hydroxyhexanoate.

Examples of polyhydroxyalkanoate homopolymers may include, but are not limited to, poly 3-hydroxypropionate (P(3HP)), poly 3-hydroxybutyrate (P(3HB)), poly 3-hydroxyvalerate (P(3HV)), poly 3-hydroxyhexanoate (P(3HHx)), poly 3-hydroxyoctanoate (P(3HO)), poly 3-hydroxydecanoate (P(3HD)), poly 3-hydroxy-5-phenylvalerate (P(3HPV)), poly 4-hydroxybutyrate (P(4HB)), poly 4-hydroxybutyrate (P(4HB)), poly 4-hydroxyvalerate (P(4HV)), or a combination thereof.

In some example embodiments, the PHA may be a copolymer (containing two or more different monomer units) in which the different monomers are randomly distributed in the polymer chain. Examples of PHA copolymers may include, but are not limited to, poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

In some example embodiments, the PHA may be a mixture of PHAs. For instance, PHA may include a mixture of a first PHA and a second PHA. The first PHA and the second PHA, for instance, may be present in the mixture at a ratio of from about 1:10 to about 10:1, such as from about 1:5 to about 5:1, such as from about 1:3 to about 3:1, such as from about 1:2 to about 2:1, or any range therebetween. The first and second PHA may be different PHAs.

For instance, the PHA mixture may include poly(3-hydroxybutyrate-co-4-hydroxyvalerate) (P(3HB-co-4HV)) and poly(3-hydroxybutyrate-co-3-hydroxyvalerate) (P(3HB-co-3HV)). The ratio of P(3HB-co-4HV) to P(3HB-co-3HV), for instance, may range from about 1:5 to about 5:1, such as from about 1:2 to about 2:1, such as from about 1:1.5 to about 1.5:1, or any range therebetween. In one example embodiment, the ratio of P(3HB-co-4HV) to P(3HB-co-3HV) may be about 1:1. In another example embodiment, the ratio of P(3HB-co-4HV) to P(3HB-co-3HV) may be about 2.5:1.

The pigment and one or more PHAs may be combined and extruded into a filament or into pellets. For instance, the pigment and one or more PHAs may be supplied either simultaneously or in sequence to a melt processing device that dispersively blends the two ingredients. In one example embodiment, batch and/or continuous melt processing techniques may be employed. For example, a mixer/kneader, Banbury mixer, Farrel continuous mixer, single-screw extruder, twin-screw extruder, roll mill, etc., may be utilized to blend and melt process the ingredients. One suitable melt processing device is a co-rotating, twin-screw extruder. Such extruders may include feeding and venting ports and provide high intensity distributive and dispersive mixing. For example, the pigment and one or more PHAs may be fed to the same or different feeding ports of a twin-screw extruder and melt blended to form a substantially homogeneous melted pigment PHA mixture.

In another example embodiment, the mixture of pigment and one or more PHAs may be processed using solvent casting to produce a pigment PHA film. For instance, the pigment and one or more PHAs may first be dissolved or suspended in a suitable solvent, selected based on the solubility characteristics of the PHAs and the desired end-use properties of the film. The pigment PHA solution or suspension can then be applied onto a flat substrate, where it is spread uniformly to achieve a consistent thickness across the surface. As the solvent evaporates, the PHA polymer matrix solidifies, and the pigment becomes embedded within it, resulting in the formation of a continuous, solid film. This process not only allows for the even dispersion of pigments throughout the PHA polymer matrix but also offers precise control over the film's thickness, texture, and surface characteristics.

Additionally, the solvent casting technique enables the customization of the film's mechanical, thermal, and optical properties, depending on the specific pigment PHA combination and processing conditions used. Variables such as the solvent evaporation rate, drying temperature, ambient humidity, and the concentration of the mixture may be adjusted to optimize the quality of the final film. The resulting film may exhibit properties such as flexibility, tensile strength, and uniform coloration, making it suitable for applications in coatings, packaging, electronics, and other industries.

In some example embodiments, the pigment produced from the engineered bacterial cell disclosed herein may be combined with PHAs for three-dimensional (3D) printing. For instance, a pigment may be extracted from engineered E. coli and combined with a resin including one or more PHAs. A mixture including extracted pigment with one or more PHAs may be stored in a container, such as ink cartridges, coupled to a 3D printer. In one example embodiment, an extracted pigment may be mixed with one or more unpigmented PHAs in a hopper prior to extrusion using a single screw extruder. For instance, an extracted pigment may be mixed with one or more unpigmented PHAs in a hopper at a temperature of from about 120° C. to about 150° C., such as from about 125° C. to about 140° C., such as from about 130° C. to about 135° C., or any range therebetween.

Various types of 3D printing techniques may be employed, such as extrusion-based systems (e.g., fused deposition modeling), powder bed fusion, electrophotography, etc. When employed in a fused deposition modeling system, for instance, the extracted pigment with one or more PHAs may be employed as the build material that forms a three-dimensional structure.

In one example embodiment, a high-resolution physical object, such as a shoe, may be formed using a 3D printer configured to deposit pigments disclosed herein layer-by-layer based on model data and/or provided computer instructions when executed by a processor of the 3D printer. In response to model data and/or computer instructions, stored in a memory, when executed by the processor, the 3D printer is controlled to successively print material from a printer head coupled to the pigment containers and moved along the gantry.

In one example embodiment, the object to be 3D printed may be footwear, such as a shoe. In another example embodiment, the objects and structures such as apparel, sports equipment, wearable devices, upholstery, jewelry and cosmetics, etc., for instance, without significant modifications.

In some example embodiments, a 3D printing system may be utilized that captures application of natural pigments and structural coloration to a printing process for objects such as objects of footwear, apparel, footwear components (e.g., upper, sole, cleat, lace, attachment), apparel components, sports equipment (e.g., balls, protective gear, pads), sports equipment components, wearable devices, upholstery, jewelry, cosmetics, and other products, among others.

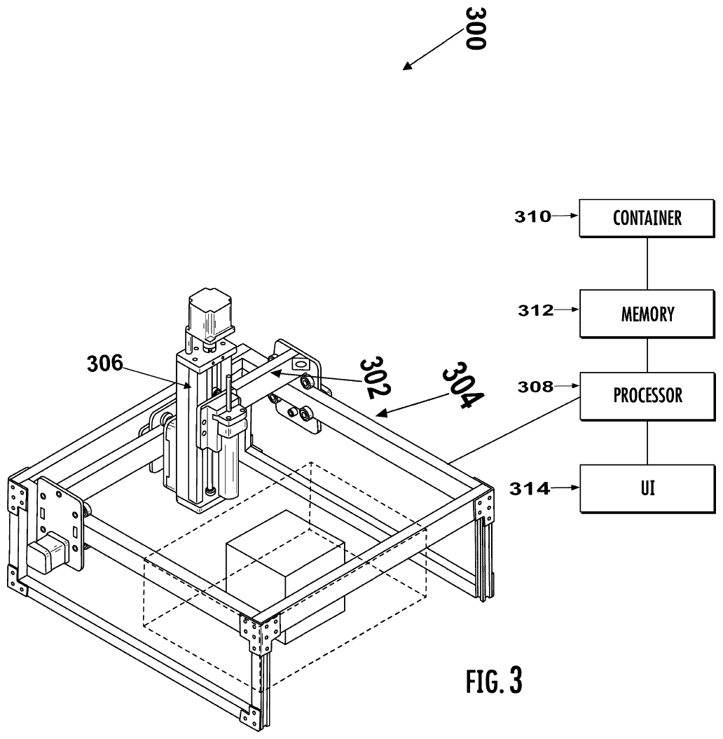

Turning to FIG. 3, solely for illustrative purposes only, a 3D printer may have a setup showing printer 300 having a movable arm 302 that slides along tracks or gantry 304 in a first direction. An injector or printer head 306 is movably attached to the movable arm 302 and is movable in a second direction which is orthogonal to the first direction. The movable arm 302 and injector/printer head 306 are moved as actuated by stepper motors in x, y, and z directions, under the control of a processor 308 which also controls the flow of material from material and/or pigment source, such as container 310 through the pneumatically actuated injector 306 to form an object by successively printing layers.

FIG. 3 also shows a memory 312, a user interface (UI) 314 operationally coupled to the processor 308 (e.g., a controller), which are all operationally coupled to the 3D printer 300. The UI 314 may include a user input device such as keyboard, mouse, touchscreen and the like, and a user output device such as display, for example. The memory 312 may store computer instructions, algorithms, software modules, and/or computer programs, as well as various data related to the object to be printed such as type and color of material along with spatial positions thereof for printing or injecting desired material and desired pigment PHAs at desired locations over a fabric base, for example. Accordingly, the memory 312 may be viewed as a data source for providing data related to the object to be printed, such as per voxel basis for example, defining size, shape, color and location of each voxel. The computer instructions stored in the memory 312, when executed by the processor 308, cause the processor 308 to control the 3D printer 300 to perform operational acts such as moving the injector 306 to a desired position and causing the release and injection of desired material and pigment PHAs to print voxels (successively or simultaneously) and/or successive layers at desired locations specified by object data from the data source or memory 312 for forming the object, such as a shoe having portions with particular patterns and colors.

The various components of the system may be operatively coupled to each other via wired or wireless connections, such as Bluetooth™, Wi-Fi™ or any other radio frequency (RF) link, for example.

The processor 316 may be a singular processor or a collection of distributed processors, such as having processors and/or controllers included with various system elements where, for example, the containers 310 and UI 314 may have their own dedicated processor(s) that, collectively with other distributed processors of system.

The memory 312 may be any type of device for storing application data as well as other data related to the described operation or system. The computer instructions and other data, such as material data of a data source and pigment data of a pigment source stored in the memory 312 are received by the processor 308 for configuring (e.g., programming) the processor 308 to perform operation acts in accordance with the present system. The processor 308 is configured to become a special purpose machine particularly suited for performing operations, acts and functions in accordance with embodiments of the present system.

The operational steps or acts may include configuring the 3D printer system 300 by, for example, controlling the injector or printer head 306 and the pigment cartridges or containers 310 in accordance with system settings.

A method of 3D printing of an object may include a step of supplying a supply source material to a print head of the 3D printer for printing a base object. As desired, portions of the base object may be scoured to encourage dye quality and colorfastness, and then may undergo mordanting to prepare the fibers to accept color. The supply source, for instance, may include a pigment PHA mixture including an extracted pigment disclosed herein and PHA. The supply source may be supplied to the print head of the 3D printer 300 for coloring a portion of a base object. In particular, the extracted pigment may be ejected from the print head and deposited on base textile object, such as when the base textile object is in a flat or planar configuration.

The methods of the present system may be particularly suited to be carried out by a computer software program, such as program containing modules corresponding to one or more of the individual steps or acts described and/or envisioned by the present system. Such program may of course be embodied in a computer-readable medium, such as an integrated chip, a peripheral device or memory, such as the memory or other memory coupled to the processor.

The program and/or program portions contained in the memory may configure the processor to implement the methods, operational acts, and functions disclosed herein. The memories may be distributed, for example between the clients and/or servers, or local, and the processor, where additional processors may be provided, which may also be distributed or may be singular. The memories may be implemented as electrical, magnetic, or optical memory, or any combination of these or other types of storage devices. Moreover, the term “memory” should be construed broadly enough to encompass any information able to be read from or written to an address in an addressable space accessible by the processor. With this definition, information accessible through a network is still within the memory, for instance, because the processor may retrieve the information from the network for operation in accordance with the present system.

The processor may be operable for providing control signals to the 3D printer and/or performing operations in response to input signals from the user input device of the UI as well as in response to other devices of a network and executing instructions stored in the memory. The processor may include one or more of a microprocessor, an application-specific or general-use integrated circuit(s), a logic device, etc. Further, the processor may be a dedicated processor for performing in accordance with the present system or may be a general-purpose processor where only one of many functions operates for performing in accordance with the present system.

The processor may operate utilizing a program portion, multiple program segments, or may be a hardware device utilizing a dedicated or multi-purpose integrated circuit.

Embodiments of the present system may provide 3D printing methods using natural pigments to form products or objects that are ecologically friendly.

The preceding description is exemplary in nature and is not intended to limit the scope, applicability or configuration of the disclosure in any way. Various changes to the described embodiments may be made in the function and arrangement of the elements described herein without departing from the scope of the disclosure.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in the compositions.

Unless otherwise indicated, all numbers expressing quantities of ingredients, properties such as percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

### EXAMPLES

### Example 1

### Genetic Engineering

E. coli strain BL21 was transformed with either pGibson-puc19-indigo or pGibson-puc19-tyrosine to make the strains E. coli tna-fmo or E. coli tyr1. Both plasmids are maintained with ampicillin resistance. Both Tna-Fmo and Tyr1 have an upstream lac promoter allowing for IPTG based induction.

### Example 2

### Pigment Production

Engineered E. coli tna-fmo or E. coli tyr1 bacteria were cultured in Luria Broth (LB) media supplemented with either 1 g/L l-tyrosine and 20 μM CuSO₄ for melanin production, 2.5 g/L tryptophan for indigo production or 1.5 g/L tryptophan and 6.3 mM cysteine for indirubin production. LB for all strains is supplemented with 100 mg/L Carbenicillin for plasmid maintenance. Pigment producing strains are cultured at 37° C. until they reach an optical density (OD₆₀₀) of 0.6-0.9. Gene expression is then induced by adding 1 mM IPTG and the culture is allowed to develop pigment over 24-48 hours with shaking at 30° C.

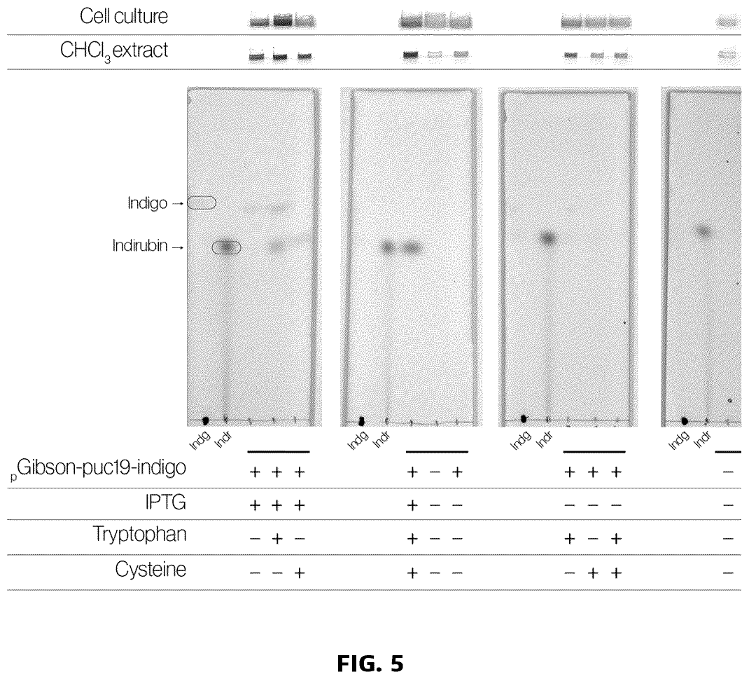

FIG. 5 depicts thin layer chromatography of indigo and indirubin cultures. Spotted samples were separated by a 50:50 hexanes and ethyl acetate mixture. Cells were induced with 1 mM of IPTG. Tryptophan was added to media at a concentration of 6.1 mM and cysteine at 3.1 mM to supplement and increase pigment production.

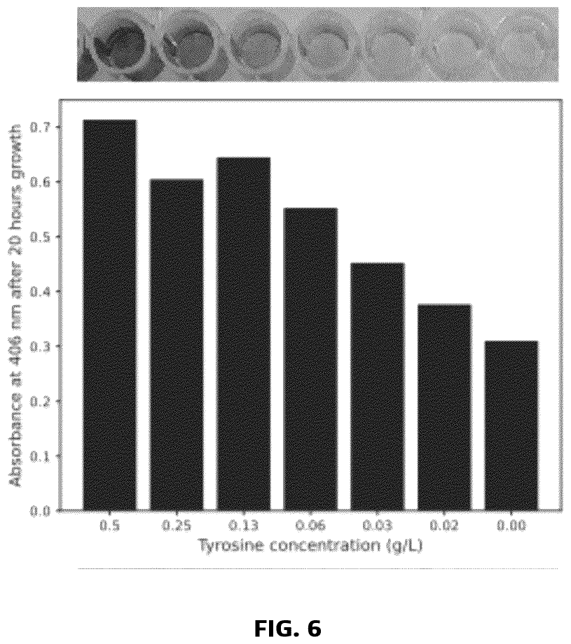

FIG. 6 depicts dependency of melanin production from E. coli pGibson_puc_tyr1 on tyrosine concentration. E. coli ₚGibson_puc_tyr1 were grown and induced with IPTG at 1 mM in M9 minimal media to show the dependency on tyrosine concentration. Cultures included tyrosine at a concentration ranging from 0 g/L to 0.5 g/L tyrosine. Data show absorbance at 406 nm after 20 hours of growth and melanin accumulation at 37° C.

### Example 3

### Pigment Extraction and Masterbatch Compounding

To extract indirubin from the pigment culture, the cells were first pelleted and then resuspended in 1% SDS. The suspension was sonicated for 90 seconds to lyse the cells. The lysed cell culture was then mixed with ethyl acetate in a separatory funnel, allowing the indirubin to move into the non-polar, organic phase, which is then separated from the liquid phase. The ethyl acetate and indirubin mixture was allowed to evaporate, leaving behind an indirubin powder. Alternatively, chloroform can be used in place of ethyl acetate. In this case, the final chloroform and indirubin mixture can be used to solubilize P(3HB-co-4HB)(4HB=30%) (CJbio, South Korea). This mixture can be solvent casted to produce a film of PHA-indirubin blend masterbatch.

To extract indigo from the pigment culture, the cells were first pelleted and then resuspended in 1% SDS. The suspension was sonicated for 90 seconds to lyse the cells. The lysed cell culture was then mixed with chloroform in a separatory funnel, allowing the indigo to move into the non-polar phase, which is then separated from the liquid phase. The chloroform and indirubin mixture was allowed to evaporate, leaving behind an indigo powder. The final chloroform and indigo mixture were used to solubilize P(3HB-co-4HB) (4HB=30%) (CJbio, Seoul, South Korea). This mixture can be solvent casted to produce a film of PHA-indigo blend masterbatch.

Melanin from the pigment culture was found both intra and extracellularly. To purify the extracellular melanin, the cells were first pelleted, and the supernatant was acidified to a pH below 2 using HCl, causing the melanin to precipitate. The precipitated melanin was separated from the acidified culture by centrifugation, and the resulting pellet was washed with water, re-acidified with HCl, re-pelleted and finally resuspended with water. To extract the intracellular melanin, the centrifuged cell pellet was then mixed with 1% SDS and sonicated for 90 seconds. The mixture was acidified to a pH below 2, followed by washing with water and re-precipitation as described above.

The final melanin-water mixture was then combined with a medium chain length PHA:water (50:50) dispersion (Terre Verdae Bioworks, Edmonton, Canada) and left to dry, to form a solution casted mPHA sheet. This mPHA-melanin sheet then is melt-compounded at 90° C. with amorphous PHA (P(3HB-co-4HB)(4HB=30%)) at ratios between 10-30% mPHA-melanin to amorphous PHA, with 3 minutes of mixing at 50 RPM in a twin-screw extruder, to create a melanin/PHA masterbatch. This masterbatch can then be mixed with pure PHA pellets using the melt extrusion method for 3D printing.

### Example 4

### 3D Printing

Pigment masterbatches are cut into pellets of a similar size to the unpigmented PHA used in 3D printing. These pigmented pellets are then mixed with unpigmented PHA in a hopper before extrusion using a single screw extruder at temperatures between 140° C. and 180° C. A gradient in pigment intensity can be achieved by varying the concentration of pigment masterbatch pellets in the hopper from the start to the end of the print.

### Example 5

### Direct Bacterial Application

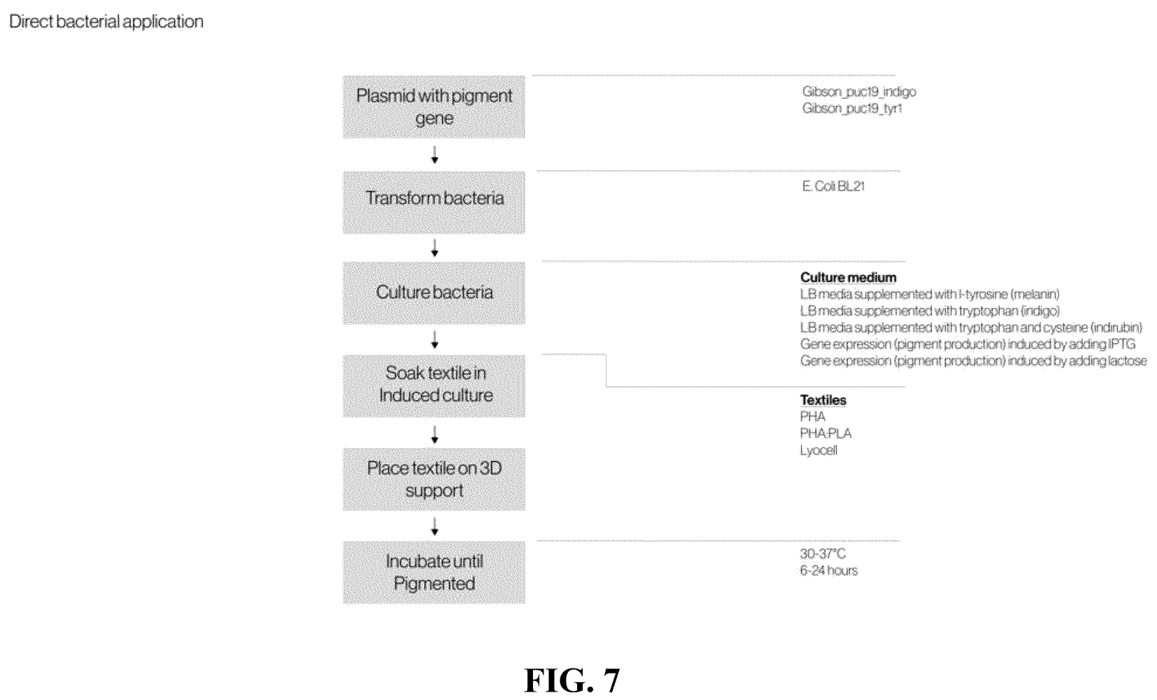

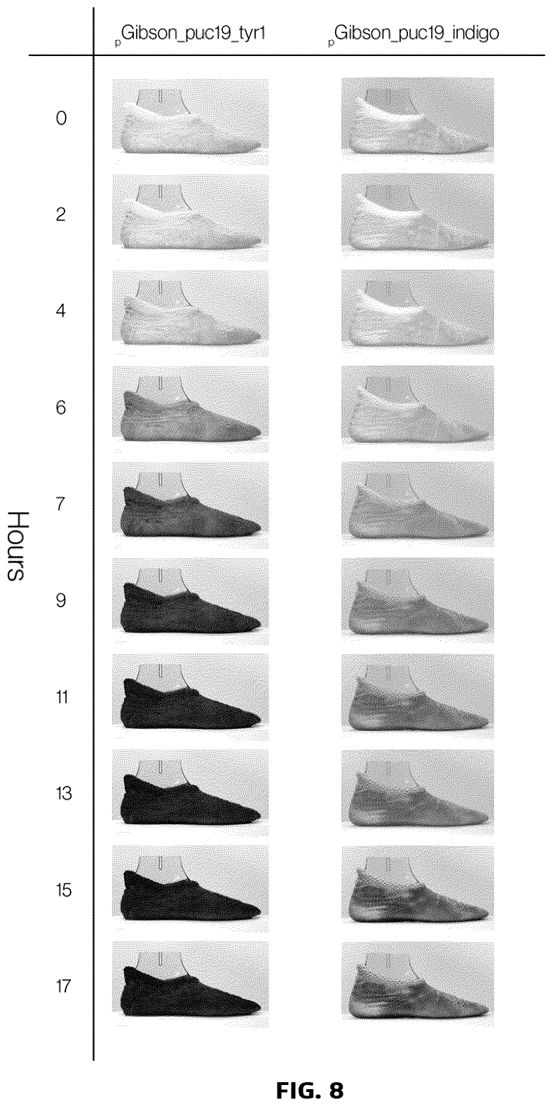

Engineered pigment-producing E. coli can be cultured directly on the surface of finished PHA products, allowing for the deposition of pigment directly on the final material (FIG. 7). Fixing bacterial cultures to the surface of PHA products may be done with physical methods, such as using thickeners and gelling agents like agar and xanthan gum, or biological methods, such as engineering pigment formation into biofilm-forming bacteria that adhere to PHA. In FIG. 8, socks were soaked in induced cultures of either E. coli pGibson_puc19_indigo or E. coli pGibson_puc19_tyr1 and imaged over time as pigmentation develops. This approach enables the application of synthetic biology techniques to PHA material.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method for producing a filament, the method comprising:
providing an engineered bacterial culture comprising a pigment;
extracting the pigment from the engineered bacterial culture by contacting the engineered bacterial culture with a solvent mixture; and
mixing the extracted pigment with a polyhydroxyalkanoate (PHA) to form a pigment PHA mixture; and
extruding the pigment PHA mixture as a filament.

2. The method of claim 1, wherein the bacterial culture is a strain selected from a group consisting of Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinomycetemcomitans, cyanobacteria, Escherichia coli, Helicobacter pylori, Selenomonas ruminantium, Shigella sonnei, Zymomonas mobilis, Mycoplasma mycoides, Treponema denticola, Bacillus thuringiensis, Staphylococcus lugdunensis, Leuconostoc oenos, Coryne bacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus cereus, Bacillus popilliae, Synechocystis strain PCC6803, Bacillus liquefaciens, Pyrococcus abyssi, Selenomonas ruminantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis.

3. The method of claim 2, wherein the bacterial culture is an E. coli strain.

4. The method of claim 1, wherein the pigment comprises indigo.

5. The method of claim 1, wherein the pigment comprises indirubin.

6. The method of claim 1, wherein the pigment comprises melanin.

7. The method of claim 1, wherein the solvent comprises water, acetone, petroleum, ether, chloroform, ethanol, ethyl acetate, or a combination thereof.

8. The method of claim 1, wherein the solvent comprises chloroform.

9. The method of claim 1, wherein the solvent mixture comprises the pigment and a solvent at a ratio of from about 1:5 to about 5:1.

10. The method of claim 1, PHA is selected from a group consisting of poly 3-hydroxypropionate (P(3HP)), poly 3-hydroxybutyrate (P(3HB)), poly 3-hydroxyvalerate (P3HV)), poly 3-hydroxyhexanoate (P(3HHx)), poly 3-hydroxyoctanoate (P(3HO)), poly 3-hydroxydecanoate (P(3HD)), poly 3-hydroxy-5-phenylvalerate (P(3HPV)), poly 4-hydroxybutyrate (P(4HB)), poly 4-hydroxybutyrate (P(4HB)), poly 4-hydroxyvalerate (4HV), or a combination thereof.

11. The method of claim 1, PHA is selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

12. The method of claim 1, wherein the PHA is a mixture of two PHA polymers in a ratio of from about 1:5 to about 5:1.

13. The method of claim 1, wherein the PHA is a mixture of poly(3-hydroxybutyrate-co-4-hydroxyvalerate) (P(3HB-co-4HV)) and poly(3-hydroxybutyrate-co-3-hydroxyvalerate) (P(3HB-co-3HV)).

14. A three-dimensional printing system comprising:
a supply source comprising a pigment polyhydroxyalkanoate (PHA) mixture, wherein the pigment PHA mixture comprises an extracted pigment produced from an engineered bacteria cell mixed with a PHA; and
a printer head that is configured to receive the polymer composition from the supply source and deposit the pigment PHA mixture onto a substrate.

15. The system of claim 14, wherein the supply source is a printer cartridge comprising a filament, wherein the filament comprises the pigment PHA mixture.

16. The system of claim 14, wherein the supply source is a hopper comprising pellets, wherein the pellets comprise the pigment PHA mixture.

17. The system of claim 14, wherein the pigment comprises indigo.

18. The system of claim 14, wherein the pigment comprises indirubin.

19. The system of claim 14, wherein the pigment comprises melanin.

20. The system of claim 14, PHA is selected from a group consisting of poly 3-hydroxypropionate (P(3HP)), poly 3-hydroxybutyrate (P(3HB)), poly 3-hydroxyvalerate (P3HV)), poly 3-hydroxyhexanoate (P(3HHx)), poly 3-hydroxyoctanoate (P(3HO)), poly 3-hydroxydecanoate (P(3HD)), poly 3-hydroxy-5-phenylvalerate (P(3HPV)), poly 4-hydroxybutyrate (P(4HB)), poly 4-hydroxybutyrate (P(4HB)), poly 4-hydroxyvalerate (4HV), or a combination thereof.

21. The system of claim 14, PHA is selected from a group consisting of poly 3-hydroxybutyrate-co-3-hydroxypropionate (P(3HB-co-3HP)), poly 3-hydroxybutyrate-co-4-hydroxybutyrate (P(3HB-co-4HB)), poly 3-hydroxybutyrate-co-4-hydroxyvalerate (P(3HB-co-4HV)), poly 3-hydroxybutyrate-co-3-hydroxyvalerate (P(3HB-co-3HV)), poly 3-hydroxybutyrate-co-3-hydroxyhexanoate (P(3HB-co-3HHx)) and poly 3-hydroxybutyrate-co-5-hydroxyvalerate (P(3HB-co-5HV)).

22. The system of claim 14, wherein the PHA is a mixture of two PHA polymers in a ratio of from about 1:5 to about 5:1.

23. The system of claim 14, wherein the PHA is a mixture of poly(3-hydroxybutyrate-co-4-hydroxyvalerate) (P(3HB-co-4HV)) and poly(3-hydroxybutyrate-co-3-hydroxyvalerate) (P(3HB-co-3HV)).

24. The system of claim 14, wherein the substrate is an object.

25. The system of claim 24, wherein the object is selected from a group consisting of footwear, apparel, sports equipment, wearable devices, upholstery, jewelry, and cosmetics.
